(19) United States
(12) Patent Application Publication
(10) Pub. No.: US 2026/0007319 A1
(43) Pub. Date: Jan. 8, 2026

(54) ELLIPTICAL FINGER CUFF

(71) Applicant: Becton, Dickinson and Company, Franklin Lakes, NJ (US)

(72) Inventor: John Randolph Dewing, Costa Mesa, CA (US)

(21) Appl. No.: 19/328,074

(22) Filed: Sep. 12, 2025

Related U.S. Application Data
(63) Continuation of application No. PCT/US2024/019200, filed on Mar. 8, 2024.
(60) Provisional application No. 63/489,853, filed on Mar. 13, 2023.

(51) Int. Cl. A61B 5/022

(52) U.S. Cl. CPC A61B 5/02241; A61B 2562/0247

(57) ABSTRACT

A finger cuff for optically measuring blood pressure includes a band having a closed elliptical shape, a light source within the band, and a light detector within the band. The band has a first diameter and a second diameter. The band is deformable such that the band's circumference along an interior surface is constant while the first and second diameters are variable. The light source is situated such that the light source is configured to emit light towards an interior opening of the band. The light detector is situated at a fixed non-zero angle relative to the light source with respect to a central point of the interior opening of the band such that the light detector is configured to receive the light emitted by the light source.

### CROSS-REFERENCE TO RELATED APPLICATION(S)

This application is a continuation of International Application No. PCT/US2024/019200, filed March 8, 2024, and entitled "ELLIPTICAL FINGER CUFF," which claims the benefit of U.S. Provisional Application No. 63/489,853, filed March 13, 2023, and entitled "ELLIPTICAL FINGER CUFF," the disclosures of which are hereby incorporated by reference in their entirety.

### BACKGROUND

The present disclosure relates generally to blood pressure monitoring devices and, in particular, to a finger cuff for optically measuring blood pressure.

Existing finger cuffs for optical blood pressure monitoring are typically wrapped about a patient's finger. These finger cuffs can be made in different sizing to accommodate varying finger circumferences. However, wrapping as a method of application can lead to misalignment of the optical components within the finger cuff, particularly in cases where an incorrect size of finger cuff is selected and/or the finger cuff is wrapped too tightly about the finger. Misalignment of the optical components due to incorrect sizing and/or overtight applications can negatively impact the accuracy of blood pressure readings by the finger cuff, but it may be difficult for medical personnel applying finger cuffs to reliably ascertain that an incorrect size selection and/or overtight application has occurred.

### SUMMARY

As discussed herein, a finger cuff for optically measuring blood pressure includes a band having a closed elliptical shape, a light source within the band, and a light detector within the band. The closed elliptical shape of the band is oriented along a first axis and a second axis which is orthogonal to the first axis. The band has a first diameter along the first axis and a second diameter along the second axis. The band is deformable such that a circumference of the band along an interior surface of the band is constant while the first diameter and the second diameter are variable. The light source is situated such that the light source is configured to emit light towards an interior opening of the band. The light detector is situated at a fixed non-zero angle relative to the light source with respect to a central point of the interior opening of the band such that the light detector is configured to receive the light emitted by the light source.

As further discussed herein, a finger cuff for optically measuring blood pressure includes a band having a closed elliptical shape, an LED within the band, a photodiode within the band, and a bladder. The closed elliptical shape of the band is oriented along a first axis and a second axis which is orthogonal to the first axis. The band has a first diameter along the first axis and a second diameter along the second axis. The band is deformable such that a circumference of the band along an interior surface of the band is constant while the first diameter and the second diameter are variable. The LED is situated such that the LED is configured to emit light towards an interior opening of the band. The photodiode is situated at a fixed 180 degree angle relative to the LED with respect to a central point of the interior opening of the band such that the photodiode is configured to receive the light emitted by the LED. The bladder is situated on the interior surface of the band and is inflatable.

As also discussed herein, a method of optically measuring blood pressure with a finger cuff includes deforming a band of the finger cuff along a first axis of the band by applying pressure along the first axis inward toward an interior opening of the band, thereby deforming the finger cuff along a second axis of the band which is orthogonal to the first axis. The band of the finger cuff is positioned about a finger. Pressure on the band along the first axis is released, thereby allowing the band to extend along the first axis and fit the finger. A light source of the finger cuff emits light across the interior opening of the band such that at least a portion of the light passes through the finger. A light detector of the finger cuff receives the light emitted by the light source which has passed through the finger. The finger cuff determines a blood pressure measurement within the finger.

The present summary is provided only by way of example, and not limitation. Other aspects of the present disclosure will be appreciated in view of the entirety of the present disclosure, including the entire text, claims, and accompanying figures.

### BRIEF DESCRIPTION OF THE DRAWINGS

FIG. 1 is a schematic depiction of a finger cuff.

FIG. 2 is a block diagram depicting a system for optically measuring blood pressure.

FIG. 3A is a front elevation view of a finger cuff in an initial, non-deformed state.

FIG. 3B is a front elevation view of the finger cuff of FIG. 3A in a deformed state.

FIG. 3C is a front elevation view of the finger cuff of FIG. 3A in a deformed state about a finger.

FIG. 3D is a front elevation view of the finger cuff of FIG. 3A in a partially- deformed, fitted state about a finger.

FIG. 3E is a front elevation view of the finger cuff of FIG. 3A in a fitted state about a finger with a bladder of the finger cuff inflated.

FIG. 3F is a perspective view of the finger cuff of FIG. 3A in a fitted state about a finger.

FIG. 4 is a flowchart depicting a method of optically measuring blood pressure with a finger cuff.

While the above-identified figures set forth one or more examples of the present disclosure, other examples are also contemplated, as noted in the discussion. In all cases, this disclosure presents the invention by way of representation and not limitation. It should be understood that numerous other modifications and examples can be devised by those skilled in the art, which fall within the scope and spirit of the principles of the invention. The figures may not be drawn to scale, and applications and examples of the present invention may include features and components not specifically shown in the drawings.

### DETAILED DESCRIPTION

A permanently closed elliptical finger cuff, for optically monitoring blood pressure, can help to reduce optical component misalignment, over-tight applications, and incorrect sizing. The ellipse has a long axis and a short axis, and includes a light source (such as a photodiode) and a light detector (such as a light emitting diode (LED)) at a fixed angle from each other. The shape of the elliptical finger cuff can be changed by gently pushing on the long axis of the ellipse, which serves to open the finger cuff. Once the cuff is placed over the finger, the pressure on the long axis of the ellipse can be released and the cuff returns to close to its original shape. The cuff thereby grips the patient's finger while the optical components remain at a fixed angle from each other.

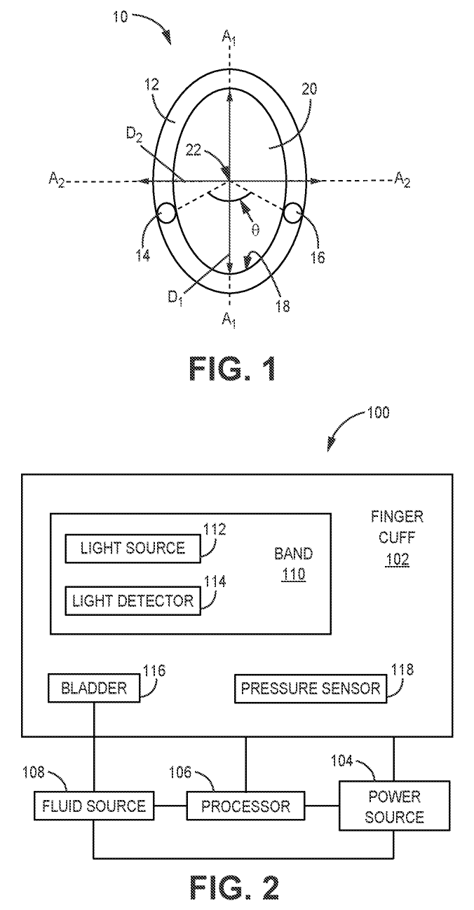

FIG. 1 is a schematic depiction of finger cuff 10. Finger cuff 10 includes band 12, light source 14, and light detector 16. Band 12 includes interior surface 18, interior opening 20, and central point 22 of interior opening 20. In some examples, finger cuff 10 can include additional components, such as a bladder which is situated on interior surface 18 of band 12 and which is inflatable. FIG. 1 also shows first axis A₁, second axis A₂, first diameter D₁, second diameter D₂, and angle 0.

Band 12 has a closed elliptical shape. The elliptical shape of band 12 can be oriented along first axis A₁ and second axis A₂. Second axis A₂ is orthogonal to first axis A₁. Band 12 has interior surface 18 along an inner circumference of band 12. Band 12 has first diameter D₁ along first axis A₁ extending between opposing sides of interior surface 18, and second diameter D₂ along second axis A₂ extending between opposing sides of interior surface 18. Band 12 is deformable, such that first diameter D₁ and second diameter D₂ are variable under applied pressure while the circumference along interior surface 18 remains constant. Finger cuff 10 is shown as being in an undeformed state in FIG. 1. Band 12 can be semi-rigid such that band 12 will hold a fixed elliptical shape (that is, having a constant first diameter D₁ and second diameter D₂) in the absence of applied pressure, and will transition back towards the original elliptical shape when pressure is released. When finger cuff 10 is situated about a finger, band 12 will transition towards the original elliptical shape until the finger is encountered. In some examples, band 12 can be formed of polycarbonate or another suitable flexible material. In some examples, band 12 can have a circumference of at least 37 millimeters. In some examples, band 12 can have a circumference of at least 43 millimeters. In some examples, band 12 can have a circumference between 37 millimeters and 43 millimeters. Light source 14 can be situated within band 12 and can in some examples be integrated into the structure of band 12. Light source 14 can be situated within band 12 and oriented toward interior opening 20 such that light source 14 can emit light towards interior opening 20 during operation of finger cuff 10. In some examples, light source 14 can be an LED. In some examples, light source 14 can be configured to emit visible light. Light detector 16 can be situated within band 12 and can in some examples be integrated into the structure of band 12. Light detector 16 can be situated within band 12 and oriented toward interior opening 20 such that light detector 16 can receive at least a portion of the light emitted by light source 14 during operation of finger cuff 10. In some examples, light detector 16 can be a photodiode. In some examples, light detector 16 can be configured to detect and/or measure visible light.

Light detector 16 can be situated at angle 0 relative to light source 14 with respect to central point 22 of interior opening 20. Angle 0 can be a fixed, non-zero angle. In some examples, angle 0 can be between approximately 160 degrees and approximately 200 degrees (that is, within approximately 20 degrees of second axis A₂). In some examples, light detector 16 and light source 14 can both be situated along second axis A₂ such that angle 0 is approximately 180 degrees. In some examples, angle 0 between light source 14 and light detector 16 can be approximately 180 degrees and light detector 16 and light source 14 are both offset from second axis A₂.

During operation of finger cuff 10, light source 14 emits light into interior opening 20 and into a patient's finger. At least a portion of the light emitted by light source 14 passes through the finger and is received by light detector 16. As described in more detail below, in order to apply finger cuff 10 onto a finger, band 12 can be deformed such that first diameter D₁ and second diameter D₂ vary while the circumference along interior surface 18 and angle 0 remain constant.

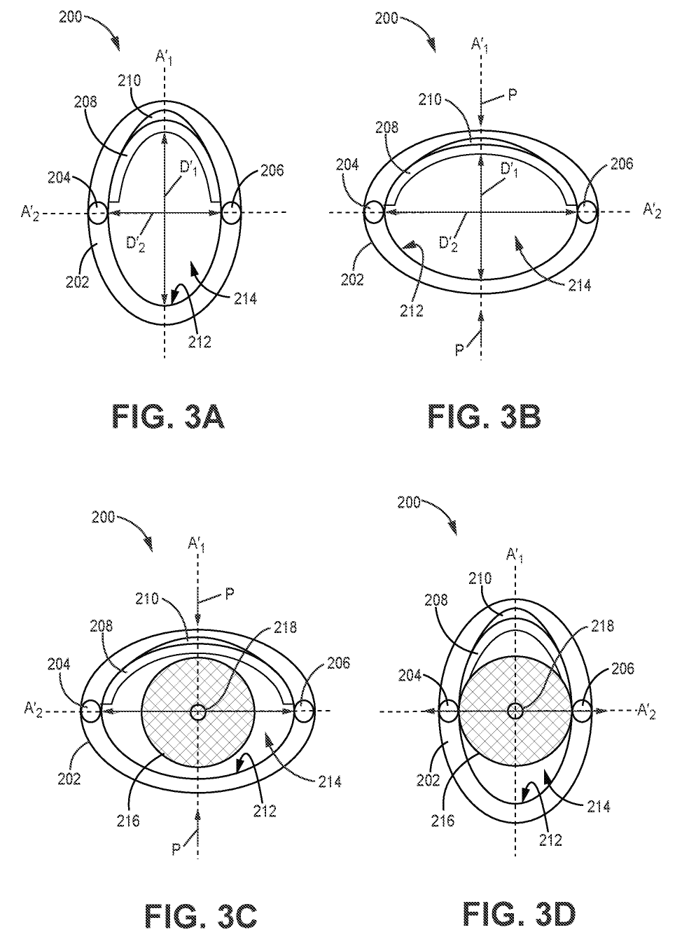

FIG. 2 is a block diagram of system 100 for optically measuring blood pressure. System 100 includes finger cuff 102, power source 104, processor 106, and fluid source 108. Finger cuff 102 includes band 110, light source 112, light detector 114, bladder 116, and pressure sensor 118.

System 100 includes finger cuff 102 that is configured to optically measure a blood pressure of a patient's finger. System 100 further includes power source 104, processor 106, and fluid source 108. Power source 104 is electrically coupled (through a wired or wireless connection) to finger cuff 102, to processor 106, and to fluid source 108. Processor 106 is also electrically coupled (through a wired or wireless connection) to finger cuff 102 and fluid source 108. Fluid source 108 is fluidly connected to bladder 116 of finger cuff 102.

Finger cuff 102 includes band 110, which includes light source 112 and light detector 114. Band 110 can be a semi-rigid, deformable band having a closed elliptical shape, such as band 12 (described above in reference to FIG. 1). Light source 112 can be any suitable light source, such as light source 14 (described above in reference to FIG. 1). Light detector 114 can be any suitable light detector, such as light detector 16 (described above in reference to FIG. 1). Finger cuff 102 further includes bladder 116 and pressure sensor 118. Power source 104 can be electrically coupled to finger cuff 102 and can provide power to light source 112, light detector 114, bladder 116, and pressure sensor 118.

Power source 104 can be electrically coupled to a finger cuff 102 and can provide power to light source 112, light detector 114, bladder 116, and pressure sensor 18. Power source 104 is also electrically coupled and can provide power to processor 106 and fluid source 108. Power source 104 can be a battery or any other suitable power source that is connected to other components by one or more wired connections and/or wireless connections. Power source 104 can be integrated into finger cuff 102 in some examples or can be a separate component in some examples.

Processor 106 can be an electronic device which can perform functions such as the detection and/or measurement of blood pressure parameters and/or the execution of instructions by components of system 100. In some examples, processor 106 can be a flexible circuit board which is integrated into, or applied onto, band 110. In some examples, processor 106 can be externally located to finger cuff 102 and can be configured to electronically communicate (through one or more wired and/or wireless connections) with components of finger cuff 102 and, optionally, other components of system 100 (such as one or more patient monitoring devices and/or other devices for displaying, recording, monitoring, and/or analyzing blood pressure parameter data).

Bladder 116 is an inflatable bladder positioned in finger cuff 102 that is configured to press against a patient's finger when inflated. Bladder 116 is fluidly connected to fluid source 108 and is thereby configured to receive a flow of fluid from fluid source 108 during operation of system 100. Fluid source 108 can supply bladder 116 with a flow of air or other suitable gas, and can additionally or alternatively supply bladder 116 with a flow of saline, hydraulic fluid, or other suitable liquid. Fluid source 108 can receive a signal from processor 106 to inflate bladder 116. In some examples, bladder 116 can receive sufficient fluid flow from fluid source 108 to withstand a pressure of between 3.5 millimeters of Hg and 44 millimeters of Hg from the finger while remaining inflated.

Pressure sensor 118 is a sensor positioned in finger cuff 102 that is configured to detect and measure the pressure applied by bladder 116 from an artery of a finger upon which finger cuff 102 is applied. Pressure sensor 118 can be one or more electronic sensors, strain gauges, mechanical sensors, magnetic sensors, optical sensors, combinations thereof, and/or other suitable sensor(s). Pressure sensor 118 is electrically coupled (through a wired or wireless connection) to processor 106 and is thereby configured to communicate with processor 106. Processor 106 can send a signal to pressure sensor 118 to sense a pressure and can receive the sensed pressure signal from pressure sensor 118.

Light source 112 can emit light into an interior opening of band 110 (such as interior opening 20, described above in reference to FIG. 1). When a patient's finger is positioned within band 110, at least a portion of the light emitted by light source 112 can pass through the finger and be received by photo detector 114. In examples where finger cuff 102 contains a bladder 116 and pressure sensor 118, pressure sensor 118 can detect and/or measure the amount of pressure applied to bladder 116 by an artery within the patient's finger. Pressure sensor 118 can, in some examples, determine the pressure applied to bladder 116 by the artery by measuring a difference between the known pressure applied to the finger by bladder 116 and the pressure and/or variations in pressure experienced by bladder 116. The blood pressure parameters detected and/or measured by photo detector 114 and pressure sensor 118 can be used to determine a blood pressure measurement of the patient.

In some examples, processor 106 can provide instructions to other components of finger cuff 102 and/or system 100. For example, processor 106 can instruct light source 112 to emit light when power source 104 supplies power to light source 112 and/or processor 106. Additionally or alternatively, processor 106 can instruct photo detector 114 to detect and/or measure the light received from light source 112. Additionally or alternatively, processor 106 can instruct fluid source 108 to inflate bladder 116 by supplying a fluid to bladder 116. Additionally or alternatively, processor 106 can instruct fluid source 108 to adjust the amount of fluid within bladder 116 in response to pressure measurements from pressure sensor 118.

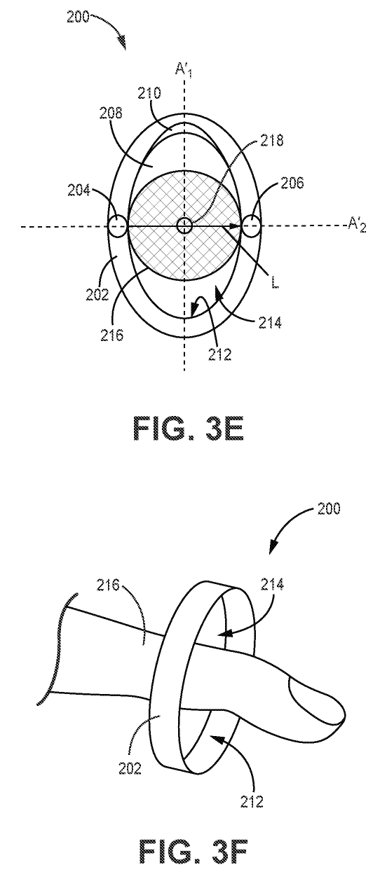

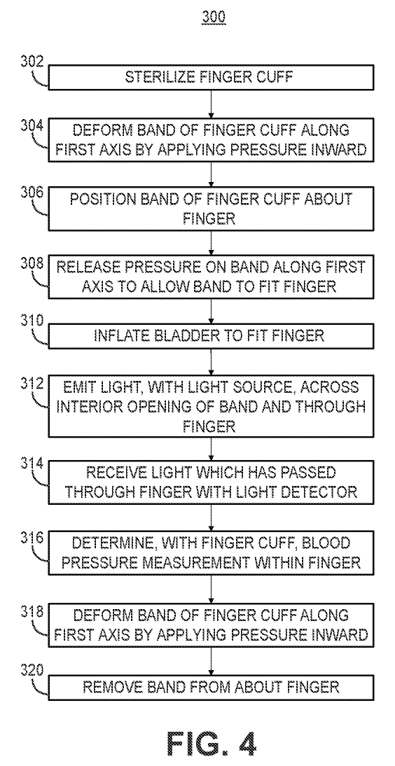

FIG. 3A is a front elevation view of finger cuff 200 in an initial, non-deformed state. FIG. 3B is a front elevation view of finger cuff 200 in a deformed state. FIG. 3C is a front elevation view of finger cuff 200 in a deformed state about finger 216 (which includes artery 218). FIG. 3D is a front elevation view of finger cuff 200 in a partially-deformed, fitted state about finger 216. FIG. 3E is a front elevation view of finger cuff 200 in a fitted state about a finger with bladder 208 inflated. FIG. 3F is a perspective view of finger cuff 200 in a fitted state about finger 216. Finger cuff 200 includes band 202, LED 204, photodiode 206, bladder 208, and flexible circuit board 210. Band 202 includes interior surface 212 and interior opening 214. FIGS. 3C-3F further show finger 216 and artery 218. FIGS. 3A-3E further show first axis A'₁ and second axis A'₂. FIGS. 3A-3B show first diameter D'₁ and second diameter D'₂. FIG. 4 depicts method 300 of optically measuring blood pressure with a finger cuff (such as finger cuffs 10, 102, and 200 described in reference to FIGS. 1, 2, and 3A-3F, respectively). Method 300 includes steps 302- 320. FIGS. 3A-3F will be discussed in turn below with the corresponding step(s) of method 300 shown in FIG. 4.

Finger cuff 200 can be used for optically measuring blood pressure within a finger. Finger cuff 200 has generally the same structure and design and can operate in substantially the same manner as finger cuffs 10 and 102 (shown in FIGS. 1 and 2, respectively) with respect to the method in which finger cuffs 10 and 102 measure blood pressure and fit a patient's finger. In the example shown in FIG. 3A, first diameter D'₁ of band 202 is larger than second diameter D'₂ when there is no pressure applied to band 202. In some examples, finger cuff 200 can include additional components, such as one or more fluid sources, pressure sensors, power sources, and/or connections for receiving power.

In step 302, finger cuff 200 can be sterilized (e.g., with heat, radiation, ethylene oxide, hydrogen peroxide, etc.) before use on a patient's finger. Step 302 can additionally or alternatively be performed after use on a patient's finger.

In step 304, band 202 of finger cuff 200 is deformed along first axis A'₁ by applying pressure inward toward interior opening 214 of band 202, as shown in FIG. 3B. This applied pressure deforms band 202 along both first axis A'₁ and second axis A'₂ that is orthogonal to first axis A'₁. FIG. 3A shows finger cuff 200 in an initial, non-deformed state, and FIG. 3B shows finger cuff 200 in a deformed state and experiencing inward pressure along arrows P. The pressure can be applied by hand by a user squeezing band 202.

In step 306, band 202 of finger cuff 200 is positioned about a finger 216 of a patient, as shown in FIG. 3C. Finger cuff 200 can be deformed sufficiently to allow band 202 to fit over the patient's finger. Deformation of band 202 shortens first diameter D'₁ of band 202 along first axis A'₁ while lengthening second diameter D'₂ of band 202 along second axis A'₂. Band 202 can be deformed to lengthen the diameter of band 202 along the shorter axis sufficiently to fit over the finger while allowing the diameter of band 202 along the longer axis to remain larger than a diameter of finger 216 along second axis A'₂.

In step 308, pressure on band 202 along first axis A'₁ is released, as shown in FIG. 3D. This allows band 202 to extend along first axis A'₁ through a lengthening of the diameter of band 202 along first axis A'₁. The extension of band 202 along first axis A'₁ can continue until band 202 fits the finger. In this manner, band 202 transitions from a fully-deformed state to a partially-deformed, fitted state (that is, partway between an initial, non-deformed state and a fully- deformed state) when pressure on band 202 is released.

In step 310, bladder 208 of finger cuff 200 can be inflated to further fit band 202 to the finger 216, as shown in FIG. 3E. Bladder 208 can be inflated by a fluid source, such as fluid source 108 (described above in reference to FIG. 2).

In step 312, LED 204 of finger cuff 200 emits light along arrow L into interior opening 214 of band 202, as shown in FIG. 3E. At least a portion of the light emitted by LED 204 passes through finger 216, including artery 218, and across interior opening 214. LED 204 can, in some examples, be configured to emit visible light.

In step 314, photodiode 206 of finger cuff 200 receives the portion of light which has passed through finger 216, as shown in FIG. 3E. Photodiode 206 can be configured to detect and/or measure the light which has passed through the finger.

In step 316, finger cuff 200 determines a blood pressure measurement within finger 216. This can be done using the detection and/or measurement of the light received by photodiode 206 in step 314. In some examples, step 316 can be performed by a processor, such as flexible circuit board 210, or processor 108 (described above in reference to FIG. 2).

In step 318, band 202 of finger cuff 200 is deformed along first axis A'₁ by applying pressure inward toward the interior opening of band 202. As in step 304 (and as shown in FIGS. 3B-3C), this applied pressure deforms finger cuff 200 along both first axis A'1 and second axis A'₂. Band 202 can be deformed to lengthen the diameter of band 202 along the shorter axis, second axis A'₂, to sufficiently to fit over finger 216 while allowing the diameter of band 202 along the longer axis, first axis A'₁, to remain larger than a diameter of finger 216, thereby allowing the removal of finger cuff 200 in step 320. The pressure can be applied by hand.

In step 320, band 202 is removed from finger 216. After band 202 is removed, pressure on band 202 along first axis A'₁ can be released, as shown in FIG. 3A. This allows band 202 to extend along first axis A'₁ through a lengthening of the diameter of band 202 along first axis A'₁. The extension of band 202 along first axis A'₁ can continue until band 202 returns to its initial, non-deformed state.

A flexible, closed-ellipse finger cuff for optical blood pressure monitoring as described herein affords numerous advantages. The shape and flexibility of finger cuff 200 allows for variable and tailored sizing for a range of finger sizes, reducing the number of sizes needed to fit expected finger size ranges. The shape and flexibility additionally reduce the likelihood of incorrect sizing and overtight applications. Further, the elliptical shape and fixed location of the optical components relative to each other helps to prevent misalignment. Finally, this finger cuff system can reduce the need for medical personnel to determine if an incorrect sizing and/or overtight application has occurred.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### DISCUSSION OF DETAILED EMBODIMENTS

The following are non-exclusive descriptions of possible examples of the present invention.

A finger cuff for optically measuring blood pressure includes a band having a closed elliptical shape, a light source within the band, and a light detector within the band. The closed elliptical shape of the band is oriented along a first axis and a second axis which is orthogonal to the first axis. The band has a first diameter along the first axis and a second diameter along the second axis. The band is deformable such that a circumference of the band along an interior surface of the band is constant while the first diameter and the second diameter are variable. The light source is situated such that the light source is configured to emit light towards an interior opening of the band. The light detector is situated at a fixed non-zero angle relative to the light source with respect to a central point of the interior opening of the band such that the light detector is configured to receive the light emitted by the light source.

The finger cuff of the preceding paragraph can optionally include, additionally and/or alternatively, any one or more of the following features, configurations and/or additional components:

A finger cuff for optically measuring blood pressure according to an example of the present invention, among other possible things, includes a band having a closed elliptical shape, a light source within the band, and a light detector within the band. The closed elliptical shape of the band is oriented along a first axis and a second axis which is orthogonal to the first axis. The band has a first diameter along the first axis and a second diameter along the second axis. The band is deformable such that a circumference of the band along an interior surface of the band is constant while the first diameter and the second diameter are variable. The light source is situated such that the light source is configured to emit light towards an interior opening of the band. The light detector is situated at a fixed non-zero angle relative to the light source with respect to a central point of the interior opening of the band such that the light detector is configured to receive the light emitted by the light source.

A further embodiment of the foregoing finger cuff, wherein the band is semi-rigid such that the band has a fixed elliptical shape in the absence of applied pressure.

A further embodiment of any of the foregoing finger cuffs, further comprising a bladder situated on the interior surface of the band which is inflatable.

A further embodiment of any of the foregoing finger cuffs, wherein the light source is an LED.

A further embodiment of any of the foregoing finger cuffs, wherein the light source is configured to emit visible light.

A further embodiment of any of the foregoing finger cuffs, wherein the light detector is a photodiode.

A further embodiment of any of the foregoing finger cuffs, wherein the fixed non- zero angle between the light source and the light detector is between 160 degrees and 200 degrees.

A further embodiment of any of the foregoing finger cuffs, wherein the fixed non- zero angle between the light source and the light detector is 180 degrees.

A further embodiment of any of the foregoing finger cuffs, wherein the light source and the light detector are situated along the second axis.

A further embodiment of any of the foregoing finger cuffs, wherein the first diameter of the band is larger than second diameter of the band where the band is in a non-deformed state.

A further embodiment of any of the foregoing finger cuffs, wherein the finger cuff is sterilized.

A system for optically measuring blood pressure includes a finger cuff. The finger cuff includes a band having a closed elliptical shape, an LED within the band, a photodiode within the band, and a bladder. The closed elliptical shape of the band is oriented along a first axis and a second axis which is orthogonal to the first axis. The band has a first diameter along the first axis and a second diameter along the second axis. The band is deformable such that a circumference of the band along an interior surface of the band is constant while the first diameter and the second diameter are variable. The LED is situated such that the LED is configured to emit light towards an interior opening of the band. The photodiode is situated at a fixed 180 degree angle relative to the LED with respect to a central point of the interior opening of the band such that the photodiode is configured to receive the light emitted by the LED. The bladder is situated on the interior surface of the band and is inflatable.

The system of the preceding paragraph can optionally include, additionally and/or alternatively, any one or more of the following features, configurations and/or additional components:

A system for optically measuring blood pressure according to an example of the present invention, among other possible things, includes a finger cuff. The finger cuff includes a band having a closed elliptical shape, an LED within the band, a photodiode within the band, and a bladder. The closed elliptical shape of the band is oriented along a first axis and a second axis which is orthogonal to the first axis. The band has a first diameter along the first axis and a second diameter along the second axis. The band is deformable such that a circumference of the band along an interior surface of the band is constant while the first diameter and the second diameter are variable. The LED is situated such that the LED is configured to emit light towards an interior opening of the band. The photodiode is situated at a fixed 180 degree angle relative to the LED with respect to a central point of the interior opening of the band such that the photodiode is configured to receive the light emitted by the LED. The bladder is situated on the interior surface of the band and is inflatable.

A further embodiment of the foregoing system, wherein the bladder is configured to receive a fluid flow from a fluid source.

A further embodiment of any of the foregoing systems, wherein the fluid flow comprises a liquid.

A further embodiment of any of the foregoing systems, wherein the fluid flow comprises a gas.

A further embodiment of any of the foregoing systems, wherein the bladder is configured to withstand a pressure of between 3.5 millimeters of Hg and 44 millimeters of Hg.

A further embodiment of any of the foregoing systems, wherein the bladder extends along at least a portion of the circumference of the band along the interior surface.

A further embodiment of any of the foregoing systems, further comprising a fluid source which is fluidly connected to the bladder.

A further embodiment of any of the foregoing systems, further comprising a power source connected to at least one of the LED and the photodiode.

A further embodiment of any of the foregoing systems, further comprising a pressure sensor which is configured to measure a pressure applied to the bladder.

A further embodiment of any of the foregoing systems, wherein the pressure sensor is a strain gauge.

A further embodiment of any of the foregoing systems, further comprising a processor.

A further embodiment of any of the foregoing systems, wherein the processor is integrated into the band of the finger cuff.

A further embodiment of any of the foregoing systems, wherein the processor comprises a flexible circuit board.

A further embodiment of any of the foregoing systems, further comprising a power source connected to at least one of: the LED, the photodiode, the bladder, and the processor.

A further embodiment of any of the foregoing systems, wherein the circumference of the band is at least 37 millimeters.

A further embodiment of any of the foregoing systems, wherein the circumference of the band is between 37 millimeters and 43 millimeters.

A further embodiment of any of the foregoing systems, wherein the band is formed of polycarbonate.

A further embodiment of any of the foregoing systems, wherein the finger cuff is sterilized.

A method of optically measuring blood pressure with a finger cuff includes deforming a band of the finger cuff along a first axis of the band by applying pressure along the first axis inward toward an interior opening of the band, thereby deforming the finger cuff along a second axis of the band which is orthogonal to the first axis. The band of the finger cuff is positioned about a finger. Pressure on the band along the first axis is released, thereby allowing the band to extend along the first axis and fit the finger. A light source of the finger cuff emits light into the interior opening of the band such that at least a portion of the light passes through the finger. A light detector of the finger cuff receives the light emitted by the light source which has passed through the finger. The finger cuff determines a blood pressure measurement within the finger.

The method of the preceding paragraph can optionally include, additionally and/or alternatively, any one or more of the following features, configurations and/or additional components:

A method of optically measuring blood pressure with a finger cuff according to an example of the present invention, among other possible things, includes deforming a band of the finger cuff along a first axis of the band by applying pressure along the first axis inward toward an interior opening of the band, thereby deforming the finger cuff along a second axis of the band which is orthogonal to the first axis. The band of the finger cuff is positioned about a finger. Pressure on the band along the first axis is released, thereby allowing the band to extend along the first axis and fit the finger. A light source of the finger cuff emits light into the interior opening of the band such that at least a portion of the light passes through the finger. A light detector of the finger cuff receives the light emitted by the light source which has passed through the finger. The finger cuff determines a blood pressure measurement within the finger.

A further embodiment of any of the foregoing methods, further comprising deforming the band about the finger along the first axis of the band by applying pressure along the first axis inward toward the interior opening of the band, thereby deforming the finger cuff along the second axis. The band is removed from about the finger.

A further embodiment of any of the foregoing methods, further comprising sterilizing the finger cuff.

A further embodiment of any of the foregoing methods, wherein determining, with the finger cuff, the blood pressure measurement within the finger comprises determining, with a processor, the blood pressure measurement.

A further embodiment of any of the foregoing methods, further comprising monitoring, with the processor, the blood pressure measurement over time so as to provide a continuous blood pressure measurement.

A further embodiment of any of the foregoing methods, wherein the processor is integrated into the finger cuff.

A further embodiment of any of the foregoing methods, wherein the processor is a flexible circuit board.

A further embodiment of the foregoing method, further comprising inflating a bladder of the finger cuff to fit the finger.

A further embodiment of any of the foregoing methods, wherein determining, with the finger cuff, the blood pressure measurement within the finger comprises determining, with a pressure sensor situated within the band, a pressure applied to the bladder by the finger.

A further embodiment of any of the foregoing methods, wherein determining, with the pressure sensor situated within the band, the pressure experienced by the bladder from the finger comprises monitoring a change in pressure applied to the bladder by the finger.

The above method(s) can be performed on a living animal or on a simulation, such as on a cadaver, cadaver heart, anthropomorphic ghost, simulator (e.g., with body parts, heart, tissue, etc. being simulated).

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

What is claimed is:

1. A finger cuff for optically measuring blood pressure, the finger cuff comprising: a band having a closed elliptical shape, the closed elliptical shape being oriented along a first axis and a second axis which is orthogonal to the first axis, the band having a first diameter along the first axis and a second diameter along the second axis, and the band being deformable such that a circumference of the band along an interior surface of the band is constant while the first diameter and the second diameter are variable; a light source within the band and situated such that the light source is configured to emit light towards an interior opening of the band; and a light detector within the band and situated at a fixed non-zero angle relative to the light source with respect to a central point of the interior opening of the band such that the light detector is configured to receive the light emitted by the light source.

2. The finger cuff of claim 1, wherein the band is semi-rigid such that the band has a fixed elliptical shape in the absence of applied pressure.

3. The finger cuff of claim 1, wherein the light source is an LED, and wherein the light detector is a photodiode.

4. The finger cuff of claim 1, wherein the fixed non-zero angle between the light source and the light detector is between 160 degrees and 200 degrees.

5. The finger cuff of claim 4, wherein the fixed non-zero angle between the light source and the light detector is 180 degrees.

6. The finger cuff of claim 5, wherein the light source and the light detector are situated along the second axis.

7. The finger cuff of claim 1, wherein the first diameter of the band is larger than the second diameter of the band when the band is in a non-deformed state.

8. The finger cuff of claim 1, wherein the finger cuff is sterilized.

9. The finger cuff of claim 1, wherein the circumference of the band along the interior surface is at least 37 millimeters.

10. The finger cuff of claim 1, wherein the circumference of the band along the interior surface is between 37 millimeters and 43 millimeters.

11. The finger cuff of claim 1, wherein the band is formed of polycarbonate.

12. The finger cuff of claim 1, further comprising a bladder situated on an interior surface of the band which is inflatable.

13. The finger cuff of claim 12, wherein the bladder extends along at least a portion of the circumference of the band along the interior surface.

14. A system for optically measuring blood pressure, the system comprising: the finger cuff of claim 12; a fluid source fluidly connected to the bladder, wherein the bladder is configured to receive a fluid flow from the fluid source, and wherein the bladder is configured to withstand a pressure of between 3.5 millimeters of Hg and 44 millimeters of Hg; a pressure sensor configured to measure a pressure applied to the bladder; a processor integrated into the band of the finger cuff, wherein the processor comprises a flexible circuit board; and a power source connected to at least one of the light source, the light detector, and the processor.

15. A method of optically measuring blood pressure with a finger cuff, the method comprising: deforming a band of the finger cuff along a first axis of the band by applying pressure along the first axis inward toward an interior opening of the band, thereby deforming the finger cuff along a second axis of the band which is orthogonal to the first axis; positioning the band of the finger cuff about a finger; releasing pressure on the band along the first axis, thereby allowing the band to extend along the first axis and fit the finger; emitting, with a light source of the finger cuff, light across the interior opening of the band such that at least a portion of the light passes through the finger; receiving, with a light detector of the finger cuff, the light emitted by the light source which has passed through the finger; and determining, with the finger cuff, a blood pressure measurement within the finger.

16. The method of claim 15, further comprising:deforming the band about the finger along the first axis of the band by applying pressure along the first axis inward toward the interior opening of the band, thereby deforming the finger cuff along the second axis; and removing the band from about the finger.

17. The method of claim 15, wherein determining, with the finger cuff, the blood pressure measurement within the finger further comprises:determining, with a processor, the blood pressure measurement.

18. The method of claim 17, further comprising:monitoring, with the processor, the blood pressure measurement over time so as to provide a continuous blood pressure measurement.

19. The method of claim 15, further comprising: inflating a bladder of the finger cuff to fit the finger.

20. The method of claim 19, wherein determining, with the finger cuff, the blood pressure measurement within the finger further comprises: determining, with a pressure sensor situated within the band, a pressure applied to the bladder by the finger.
